(19) United States
(12) Patent Application Publication
(10) Pub. No.: US 2008/0262631 A1
(43) Pub. Date: Oct. 23, 2008

(54) Method For Providing a Polymeric Implant With a Crystalline Calcium Phosphate Coating

(75) Inventors: Johannes Arnoldus Jansen, Elst (NL); Bastiaan Feddes, Utrecht (NL); Johannes Gerardus Cornelis Wolke, Druten (NL); Arjen Maarten Vredenberg, Utrecht (NL)

Correspondence Address: ABELMAN, FRAYNE & SCHWAB, 666 THIRD AVENUE, 10TH FLOOR, NEW YORK, NY, 10017, US

(73) Assignee: STITCHING KATHOLIEKE UNIVERSITEIT, Nijmegen (NL)

(21) Appl. No.: 10/586,041

(22) PCT Filed: Jan. 19, 2005
(86) PCT No.: PCT/NL2005/000038

§ 371 Date: Jul. 13, 2006

(30) Foreign Application Priority Data
Jan. 19, 2004 (EP) 04075164.6

(51) Int. Cl. A61F 2/28; A61L 27/12

(52) U.S. Cl. 623/23.58; 427/2.26

(57) ABSTRACT

This invention relates to a method for crystallizing an amorphous calcium phosphate (Cap) by high energy laser light irradiation. The method is particularly suited for crystallizing CaP coatings on polymeric substrates, particularly implant objects.

### FIELD OF THE INVENTION

This invention is in the field of ceramic coatings on implants, in particular of crystalline hydroxyapatite coatings on polymeric implants.

### BACKGROUND OF THE INVENTION

Biomaterials are used to replace parts of the body that are diseased, worn, or broken. Annually, millions of operations are performed which involve bone repair. Due to the higher life expectancy and wealth, the number of implants that are used will increase further. One of the materials that is frequently used to regenerate bone, is calcium phosphate (also abbreviated herein as CaP) ceramic. The CaP ceramic that is most frequently used is hydroxyapatite (HA, Ca₅(PO₄)₃OH). As a result of the chemical composition of this material, which is similar to the mineral component of bone, it enhances the bone formation process around the implant. CaP ceramic is therefore called ‘bioactive’. To avoid the brittle nature of bulk CaP ceramic, the material is often applied as a coating.

Usually, the CaP coatings are deposited on metallic substrates. However, a polymeric substrate may be a more suitable alternative, because the mechanical properties of a polymer can easily be varied. A polymeric substrate effectively transfers force from the implant to the surrounding bone, avoids bone resorption due to so-called ‘stress shielding’ [1]. On the other hand, most of the polymeric materials that are used for the manufacturing of implants are bioinert, i.e., do not induce bone healing. Therefore, a polymeric material, coated with a CaP coating for a better bone response, may be an interesting system for medical applications, e.g. in fixation plates or screws. The use of a CaP coating on the polymeric parts of such devices may improve the biological response. For instance, by using a degradable polymer, e.g. Poly-L-Lactic acid (PLLA), for fixation plates or screws, a second operation for the removal of the screws or the plate can be prevented. Besides for orthopaedic surgery, other applications for CaP coated polymeric implants may be found in maxillofacial surgery.

In order to use a CaP coated polymeric material for medical applications, it needs to meet some requirements. First of all, a good adhesion of the CaP coating to the polymeric substrate is needed. For example, the American Food and Drug Administration (FDA) requires a minimum tensile coating strength of 50.8 MPa [2]. Besides a good adhesion, also the coating composition has to be controlled, as a Ca/P ratio around 1.67 (the ratio of HA) is desired. Finally, a certain degree of coating crystallinity is beneficial, in order to prevent the rapid dissolution of the coating under in vivo conditions [3]. Therefore, it is an object of this invention to provide polymeric substrates with crystalline CaP coatings.

Usually, deposited CaP coatings, also radiofrequent (RF) magnetron sputter deposited CaP coatings, are amorphous. As already mentioned amorphous RF magnetron sputter deposited coatings dissolve, both under in vitro, as well as under in vivo conditions [3, 4]. Coatings which are (partially) crystalline do not show this dissolution. In principle, crystalline coatings can be obtained by annealing RF magnetron sputter deposited amorphous coatings at a temperature of at least 500° C. [5, 6, 7]. Also for CaP coatings that are deposited using other techniques, such high temperatures (400-600° C.) are needed [8, 9, 10-13].

As mentioned above typically 400-600° C. is required to transform a coating from an amorphous to a (partially) crystalline one. Unfortunately, these temperatures are too high for polymers. Nevertheless, Hontsu et al. [14, 15] investigated the crystallization of CaP coatings on polymeric substrates (PTFE (polytetrafluoroethylene), PI (polyimide), PDMS (polydimethylsiloxane) and PET (polyethtyleneterephthalate)), by performing a long anneal (10 hours) just below the melting temperature of the substrates. On PTFE and PI, partially crystalline coatings were obtained by annealing at 320° C. and 360° C. respectively. However, on PDMS and PET, the CaP coating remained amorphous, after an anneal at 240° C. and 260° C., respectively. It is clear that an annealing procedure has strong disadvantages in the case of polymeric substrates. Besides the strong difference in thermal expansion (which easily causes delamination [14]), the largest problem is that only high temperature resisting polymers can be used.

Katto et al. in Surface and Coatings Technology, vol. 169-170 (2003), p 712-715, describe a method for depositing hydroxyapatite onto a metal (Ti) substrate. An assisting laser beam is split off from the KrF laser deposition beam and is used to irradiate a metal (Ti) substrate at the same time while the coating is being deposited. This resulted in improved adhesion of the CaP coating, which is ascribed to an annealing process by the assisting laser resulting in crystallinity of the hydroxyapatite coating. Also a second ArF excimer laser was used as assisting laser for irradiation of the Ti substrate. Whereas it is suggested that crystallinity can be controlled by changing the deposition parameters, proof that a crystalline CaP coating actually has been obtained is conspicuously absent from the publication. Moreover, Katto et al. teach that deposition and assisting lasers always operate simultaneously, optionally with a time lag on the order of nanoseconds, and thus that the substrate will always be heated up by the assisting laser.

WO 94/22513 describes catheters that are provided with a CaP coating. The coating is applied using short pulsed laser deposition. It is mentioned that for temperature sensitive substrates an amorphous hydroxyapatite (CaP) coating can be crystallised by laser anneal using any suitable short pulsed laser. What in fact is shown in WO 94/22513 is laser anneal of hydroxyapatite on silicon (Si), i.e. the temperature insensitive basic material for semi-conductors, with a pulse energy of 100-200 mJ/cm² at a pulse frequency of preferably 2000. At the wavelengths of the lasers mentioned and using the high pulse frequency, the Si substrate will be heated which means that the method described in WO 94/22513 cannot be used for polymeric substrates. Further only crystalline coatings on metal substrates are disclosed at a deposition temperature of 500° C.

Antonov et al. in Lasers in the Life Sciences vol. 9(3) (2000), pp 127-142, concerns the modification of CaP coatings on Ti and Teflon substrates by pulsed laser irradiation at 213 nm. It is noted that in a relatively thick coating of 1 μm only 40% of the energy is absorbed by the CaP coating. Further the authors state that 30% of the laser radiation is reflected from the Ti substrate, which means that most of the energy is absorbed in the 1 μm CaP coating, the remainder of the energy heats up the substrate. It is concluded that because of this, for Teflon substrates the laser annealing process is not as efficient as that for metallic substrates since in the process the CaP coating is easily peeled of from the Teflon substrate. Antonov et al. then teach that the problem of peeling of from Teflon substrates is to be solved by the fine adjustment of the laser fluence.

In the remote field of semiconductor technology laser induced crystallization has been proven to be applicable for the crystallization of amorphous thin films on temperature-sensitive materials. For example Smith et al. [16], described crystallization of silicon (Si) on plastics (PET) [16, 17]. Irradiation occurred at fluencies up to 450 mJ/cm² resulting in a maximum temperature of 250° C. in the substrate material. It has to be noticed that Si is not comparable with CaP ceramic. In general, ceramic materials are composed of multicomponents. Also no optical information is available about CaP ceramics. The complexity of ceramic materials, is confirmed in studies where indium-tin oxide [18], and TiO₂, Nb₂O₅, Ta₂O₅, and SrTiO₃ [19] were crystallized onto metal substrates using KrF (248 nm) or ArF (193 nm) excimer laser pulses of ≧40 mJ/cm². These materials concern optical coatings, magnetic/polarisable coatings and hard coatings for tools. Also these examples are far removed from the field of implants and the coating of implants with biocompatible materials.

### DESCRIPTION OF THE INVENTION

It was found that CaP ceramic material has the ability to absorb UV light of short wavelength and further it was found that by using short energetic UV-laser light pulses, an amorphous CaP coating crystallises. Advantageously, the crystallisation process takes place without damaging the polymeric substrate onto which the amorphous CaP is coated to a significant extent. Without being bound by theory it is believed that wavelengths that are too long penetrate through the ceramic coating and heat subsequently the underlying substrate material instead of the coating, with concomitant detrimental effects on the temperature-sensitive polymeric substrate. CaP coatings show an optical absorption edge at 200 nm and thus the wavelength of the laser light that is used for irradiation should be les than 200 nm.

It was found that if a polymeric substrate for implantation having deposited thereon an amorphous calcium phosphate (CaP) coating was irradiated with laser light of <200 nm and with an energy between 10 and 1000 mJ/cm², the amorphous CaP coating crystallises.

Thus the invention concerns a method for providing a polymeric implant object with a crystalline calcium phosphate (CaP) coating, said method comprising the step of irradiating a polymeric substrate having deposited thereon an amorphous CaP coating with laser light of <200 nm and 10-1000 mJ/cm².

Advantageously the present method allows complete disconnection of the crystallization process from the deposition process, offering the possibility to use any process for deposition of the CaP coating. Nevertheless, it is also possible to perform the laser crystallization during the deposition process of the coating. Thus in an embodiment the invention concerns a method according to the invention in which the irradiating with laser light <200 nm and 10-1000 mJ/cm² is carried out during deposition of a CaP coating onto a polymeric substrate. An optical viewport, which transmits the appropriate laser light, should be present in the deposition system.

The implant object in the present method is a polymeric substrate, i.e. a substrate made of polymer (plastic). The term ‘polymeric substrate’ refers to a substrate made of any plastic material, or combination of materials including a plastic material, that is suitable to serve as an implant. Polymeric materials or biomaterials preferably resemble as much as possible the natural tissue in which they are intended to be inserted. Further, polymeric biomaterials must be sterilizable and tissue compatible. Depending on their application, they can be degradable. Suitable materials to include, partially or entirely, in polymeric substrates comprise at least one selected from the group consisting of polyethylene (PE), poly(ethyleneterephtalate) (PET), polytetrafluoroethylene (PFTE), polystyrene (PS), poly-L-lactic acid (PLLA), polydimethylsiloxane (PDMS), polyimide (PI), polyglycolic acid (PGA), polypropylene fumarate (PPF) and polybutylterephthalate (PBT).

Nevertheless, the present invention provides a method that is equally well suited to crystallise amorphous CaP coatings on metal implant objects or for instance implant objects of a combination of metal and temperature-sensitive material, such as for instance the polymeric materials described above

There is a variety of processes to deposit CaP coatings. In the method of the invention any process for depositing a CaP coating onto a substrate can be employed. Thus in the method of the invention the CaP coating is deposited using any method suitable for depositing a CaP coating, said deposited CaP coating being amorphous. Some of the most frequently used are:

### Plasma Spraying

Plasma spraying is a technique that is most frequently used for the application of CaP coatings [9, 10, 20]. It is based on feeding CaP particles in a carrier gas through an electric arc. The gas becomes a plasma, which is accelerated to high velocities. The particles that are transported in the carrier gas are deposited on a substrate. In this technique the substrate may rise to high temperatures, which may make it less suitable

### Biomimetic Deposition

Biomimetic coatings are formed from simulated physiological fluids. CaP ceramic is deposited on the substrates from a supersaturated solution [21,22]. An advantage of this technique is its simplicity and the possibility to coat complex geometrical shapes at ambient temperature. This makes the technique suitable for covering polymeric substrates. A disadvantage of this technique is that the adhesion of the CaP coatings, especially on inert polymeric substrates, may be poor [21].

### Laser Deposition

Laser deposition is a technique in which intense UV excimer laser pulses are used to evaporate CaP ceramic from a target [23,24]. A major advantage of this technique is that the chemical composition of the target is transferred to the coating in the deposition process [24]. As a result of the low substrate temperature during the deposition process, this technique is also suitable for the deposition of CaP coatings on polymeric substrates [14].

### Ion Beam Deposition

In ion beam deposition, a CaP target is bombarded by a beam (energies keV) from an ion gun. Particles are ejected (‘sputtered’) from the target, and deposited on the substrates [5, 25, 26]. It produces amorphous coatings, which adhere quite well to metallic substrates (10-60 MPa). However, the chemical composition of the coating may deviate from that of the sputtering target.

### RF Magnetron Sputter Deposition

RF magnetron sputter deposition is the technique that was used for the experiments described herein. The technique has some resemblance with ion beam deposition. In this method, a CaP ceramic target is irradiated with energetic particles, which cause the sputtering and subsequent deposition of the target components. The energetic projectiles that cause the sputtering are created in a plasma. Compared to the ion beam deposition technique, this method is more complex, due to the simultaneous occurrence of many processes. The way in which the deposition process works will be explained in more detail below. It has been shown that a large variety of compositions can be obtained, depending on gas composition [27], gas pressure [28], and discharge power [29]. Moreover, an excellent adhesion was found for the CaP coatings on metallic substrates [30]. An advantage of this technique is that, when the deposition power is kept low, the heat load on the substrates remains low. Further, homogeneous coatings up to several micrometers can be produced in easy operation and the reproducibility, in terms of coating composition and growth rate, is good. Another advantage is that the sputter deposition process is already used in commercial production.

In an embodiment of the invention the method suitable for depositing a CaP coating is selected from plasma spraying, biomimetic deposition, laser deposition, ion beam deposition and RF magnetron sputter deposition or combinations thereof.

In a preferred embodiment of the method of the invention the CaP coating is deposited using an RF magnetron sputter process.

In the examples a number of polymeric substrates is described which show different adhesion behaviour toward CaP ceramic. This different behaviour can greatly be understood from the interfacial structure and phenomena that occur during deposition processes. Therefore, every polymer needs its own investigation, thereby keeping in mind that coating delamination may have different origins (both interfacial or within the polymer itself). It is well within the reach of the skilled person to vary deposition parameters and arrive at a satisfactory adhesion of CaP ceramic on the plastic substrate. Some polymers (PE, PS, and PTFE) were used that were intended for research and therefore contained a minimum amount of additives. Plastics consist of polymer and additives. It should be realized that these additives may also influence the adhesion of coatings. Also it may be advantageous to subject the polymer to any kind of pretreatment. Methods of pretreatment are known in the art and include surface cleaning, ablation, crosslinking of the polymeric material, modification of the chemical structure by for instance chemical treatment, UV light treatment, corona treatment, plasma treatment and ion beam treatment and also an interlayer of reactive material, from for example Ti or Cr, may be applied.

Very good adhesion can be obtained, even on polymers that are known for their problematic adhesion behaviour (PE). A possibility may be the combination of two different deposition techniques. For example, rf magnetron sputter deposition may be used for growing the strong interfacial structure, after which the growth is continued in solution using the biomimetic technique. This may solve the adhesion problems that were reported for biomimetically grown CaP on some polymers [21].

The method of the invention can be improved by optimised deposition system design and process parameters and for instance optimal substrate cooling. This method of the invention can lead to homogeneous, dense, crystalline, well adhering CaP coatings on different substrates including polymeric substrates, which can be used for new implants.

In order to achieve crystallisation of the amorphous CaP coating it is necessary that the irradiated laser light is absorbed in the CaP ceramic material. This means that not all lasers are suitable for this purpose. FIG. 3 shows the optical transmission spectrum of CaP coated sapphire. It is noted that variation of the CaP composition, for instance by variation in the ratio Ca to P, or by the addition of additives, such as for instance titanium, the absorption of CaP ceramic material may be influenced. Nevertheless, in general the wavelength of the irradiated laser light should be less than 200 nm. Suitably a laser selected from the group consisting of F₂ (157 nm) and ArF (193 nm) is used. Preferably an F₂ laser is used.

The duration of laser light irradiation depends on the energy of the laser pulse and an energy for the laser pulse is selected dependent on the thickness of the CaP coating. Variation of these parameters so as to come to a suitable energy for the laser pulse and irradiation with this energy for a suitable period of time, given a desired thickness of the CaP coating, is well within the ambit of the skilled person.

Preferably the method of the invention is used to crystallise thin coatings, i.e. coatings of not more than 10 μm. Coatings that can suitably be crystallised may be as thin as several nm; for instance 10 nm, preferably from 50 nm and higher. In an embodiment the thickness of the coating ranges from 50 nm to 5 μm. In another embodiment the thickness of the coating ranges from 100 nm to 4 μm. Advantageously very thin coatings can be crystallised such as coatings of less than 1 μm and even less than 0.5 μm. In further embodiments the thickness of the coatings ranges from 10 nm to less than 1 μm or 10 nm to less than 500 nm, or 100 nm to less than 1 μm, or 100 nm to less than 500 nm or 50 nm to 400 nm.

In general irradiation with an energy of less than 10 mJ/cm² does not lead to sufficient crystallisation, whereas generally irradiation with more than 1000 mJ/cm² will lead to unacceptable delamination. In one embodiment the energy of the laser pulse ranges from 10 to 500 mJ/cm². In one embodiment the energy of the laser pulse ranges from 10 to 250 mJ/cm².

The duration of the laser pulse may range from 1 to 50 ns. It is also possible to irradiate a certain area of a substrate repeatedly. Further optimalisation of the method of the invention may be realised for instance by variation of the area or spot size that is irradiated with a pulse, pulse frequency and number of pulses.

An additional advantage of the use of a laser to crystallise the CaP coating is the accuracy with which the irradiation can be controlled. In particular the specific position of the laser relative to the object to be irradiated can be controlled, which allows the laser to move across the object to be irradiated along discrete paths thereby creating a certain pattern of crystallisation on the irradiated object. This may be advantageous for creating patterns in coatings on implant objects that have optimal interaction with the structure of the bodily material or tissue they are implanted in. In particular patterns that orient cellular behaviour are advantageous and such patterns are known to a person skilled in the art. Examples of suitable patterns are grooved or square shaped patterns.

Thus in a further embodiment of the method of the invention a pattern is made in the crystalline CaP coating.

In a further aspect the invention relates to polymeric implant objects that are obtainable by the method according to the invention. Examples of implant objects according to the invention are fixation plates, fixation screws, medullary nails, acetabular cups, guided tissue regeneration membranes. In one embodiment the invention concerns a polymeric implant object, said object comprising a polymeric substrate having a crystalline CaP coating, said crystalline CaP coating having a thickness of at least 10 nm, but less than 1000 nm. In other embodiments it is preferred the coating is at least 50 nm or at least 100 nm or at least 200 nm thick but less than 900 nm or 800 nm. For very thin coatings in one embodiment the coating is less than 500 nm thick. Advantageously the crystalline coating can be applied onto flexible polymeric implants; due to the cristallinity of the coating it is allowed to bend or fold the flexible implant without damaging the CaP coating. Thus in one embodiment the polymeric implant object comprises or is made of flexible polymeric material.

### DESCRIPTION OF THE FIGURES

FIG. 1: A schematic drawing of the deposition chamber. The left figure is a front view, and the right figure is a top view. The cross (×) and the star (★) denote the positions of the substrates when a static deposition is performed.

FIG. 2: The meandering track that was used for the laser irradiation experiment. The dots indicate the centers of the successive laser spots, while the dotted squares (Sx×Sy) indicate the spot size.

FIG. 3: The optical transmission of CaP coated and uncoated sapphire. The wavelengths of different excimer laser are also indicated.

FIG. 4: XRD spectra of as-deposited CaP on PE (A) and F₂-laser treated CaP on PE (B, C and D). The energy per laser pulse was 60, 96, and 200 mJ/cm2 for B, C, and D respectively. The positions of several HA reflections are also indicated.

FIG. 5: SEM images of F₂ laser irradiated CaP on PE. The energy per pulse was, for A-G, 0, 6, 10, 18, 49, 69, and 200 mJ/cm², respectively. H is an enlargement of F.

FIG. 6: Remaining coating (in percent) after laser irradiation with different intensities.

FIG. 7: Calculated temperature distribution in the CaP coating after a laser pulse of 60 mJ/cm² (A) and 200 mJ/cm² (B).

### EXAMPLES

### Experimental Techniques

### RF Magnetron Sputter Deposition

Sputter deposition is based on the ejection of species from a target by highly energetic particles. The energetic projectiles can be generated using an ion gun or plasma. The ejected species end up on the substrates to form a coating.

In RF sputter deposition, the projectiles are generated by an RF plasma. An RF instead of a DC field is often used in case of an insulating target, to avoid charging and arcing. Electrons that follow the alternating RF field cause ionizations of the gas in the vacuum chamber. Positive ions that form in the plasma (often argon ions) are accelerated when they cross the so-called plasma sheaths, which is a sudden drop in potential at the edges of the plasma. The accelerated ions cause the ejection of species from the sputter target and they also initiate the ejection of secondary electrons, which are responsible for additional ionizations in order to maintain the plasma.

The sputtering rate of an RF sputter deposition system can be greatly enhanced by applying a magnetron configuration below the sputtering target. The magnetic field traps the electrons nearby the target. The trapped electrons cause additional ionizations in the gas, enhancing the sputter rate.

### Deposition System

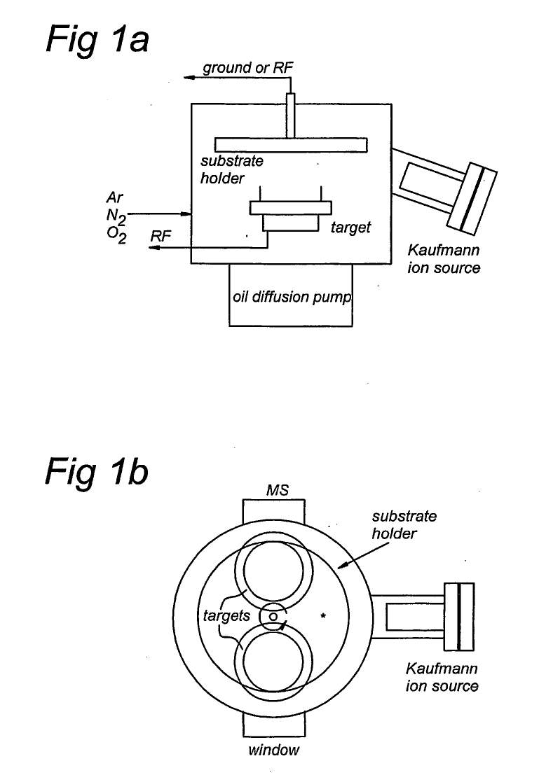

The pretreatment of the substrates and the deposition of the coatings were conducted in an Edwards High Vacuum ESM100 sputter deposition chamber. A schematic drawing of the system is given in FIG. 1. The system consists of a stainless steel chamber with a diameter of 32 cm. A base pressure of 1×10.⁻⁵ mbar is reached using an oil diffusion pump. The water-cooled substrate holder is located in the top part of the ground or RF deposition chamber and can be used in a static or rotating (30 rotations per minute) configuration. For static depositions, the samples are either mounted directly above the target or at the focal point of the ion gun. These positions are denotes in FIG. 1 by × and [symbol] , respectively. For rotating depositions, the samples are mounted 7±1 cm from the centre of the substrate holder (which means that they pass x and [symbol] during rotation). The two sputtering targets are about 8.2 cm below the substrate holder. They are connected to separate power supplies that operate at 13.56 MHz. The targets have a grounded shield and cross contamination shields on top. During static depositions, the cross contamination shields are removed to increase the deposition rate at the focal point of the ion gun [symbol] . Below the sputter targets, magnetron configurations are present.

Under normal deposition conditions, the substrate holder is grounded. However, it is also possible to use the substrate holder as a powered electrode by connecting one of the target power supplies to it. In this way, samples are pretreated, for example by oxygen bombardment. The discharge gas is led into the deposition chamber via a piezo electric valve, which is coupled to a pressure gauge. A feedback loop in this system ensures a constant gas pressure during depositions.

A Kaufmann ion source (Oxford Applied Research model RF 25) is connected to the deposition system. The gun can either be used for pretreatment of materials or for ion beam assisted deposition. The focus of the gun is approximately 7.5 cm from the centre of the substrate holder and the gun is mounted at an angle of 70°, relative to the normal of the substrate holder.

Finally, a Quadrupole Mass Spectrometer (MS) (Vacuum Generators model Sensorlab) is connected to the system. This instrument can be used for measuring the sputtering gas composition as well as detecting contaminations in the sputtering gas.

### The Substrates

Substrate materials used were silicon single crystals (orientation (100), obtained from Gritek Ltd.); polystyrene (PS), polyethylene (PE), and polytetrafluoroethylene (PTFE, teflon) sheets (thickness 1 mm) were obtained from Goodfellow Ltd; polydimethylsiloxane (PDMS, silicone rubber) was obtained from Viba NV (Elastosil RT 601). The material was polymerized in a plastic box. The smooth air cured side was used. Poly-L-lactic acid (PLLA) granules were obtained from Purac Biochem. The granules were transformed in PLLA plate (thickness 1 mm) by heating the material in a steel mould to 170-180.C.

Prior to the deposition or pretreatment, the substrates were ultrasonically cleaned in isopropanol for five minutes.

### Coating Deposition

For the sputtering target, hydroxyapatite (Ca₅(PO₄)₃OH) was used, either, in the form of small granules (diameter 0.5-1.0 mm) or plasma sprayed on a copper disk. The target was 10 cm in diameter. Usually, only one target was used during deposition. To further reduce the heat load on the samples, the power supply was operated at a maximum power of 200 W. Above this power, softening of the substrates started to occur (especially PS). A typical deposition rate at a deposition power of 200 W was 1-2 nm per minute. Usually, the sputtering gas was argon. The pressure during coating deposition was between 5×10.⁻⁴ and 1×10.⁻¹ mbar.

### Pretreatment

As already mentioned, a pretreatment of polymers prior to coating deposition may increase the adhesive strength. In particular for pretreatment of polymeric samples plasma pretreatment and ion beam treatment have been used successfully.

### Plasma Pretreatment

The plasma pretreatment can be done by changing the normally grounded substrate holder into a RF powered electrode. A typically used plasma pretreatment was the exposure of the polymeric substrates to an oxygen plasma of 2×10.3 mbar, at a power of 200 W, for 30 sec.

### Ion Beam Pretreatment

An ion bombardment is a better defined pretreatment. The ion current as a function of gas pressure and ion gun power level was measured using a Faraday cup. The optical emission of the plasma in the ion gun was measured during operation in order to check plasma stability and plasma mode (low brightness or high brightness mode [31]). The energy of the ions could be varied between 100 and 1000 eV. The beam shape is almost Gaussian and has a full width at half maximum (FWHH) of 1.5 cm. For the experiments in this study, argon gas was used for the ion gun.

### Coating Analysis

X-Ray Diffraction (XRD)

X-ray Diffraction (XRD) is a technique to determine the crystallinity of the coating. A thin film Philips PW3710 X-Ray Diffractometer, equipped with a Cu-K\ddot{α} source, was used. The angle of incidence was fixed at 2.5., while the solid state detector was scanned.

### Coating Deposition & Analysis

PE was used as substrate. For the deposition, the plasma sprayed HA target was used and the substrate holder was rotated during the deposition process. The deposition time was 3.5 hours, the power was 200 W, and the Ar pressure was 5.0-10.⁻³ mbar. The resulting Ca/P ratio amounted to ˜1.6 and the coating thickness was ˜250 nm, as determined by Rutherford Backscatter Spectroscopy (RBS).

To determine the optical absorption of the CaP coating, the optical transmission of uncoated sapphire (Single Crystal Technology, The Netherlands) and sapphire with ˜1 μm CaP coating was measured. For this, a UV-VIS apparatus (UVIKON 923) was used, between 190 and 900 nm.

### Laser Annealing

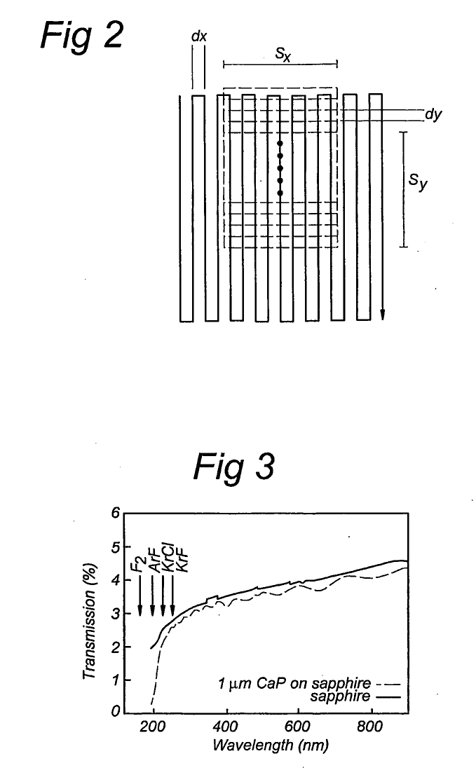

The Lambda Physik LPF-220 fluorine laser of the Fraunhofer Institute for Laser Technology (Aachen, Germany) was used to irradiate the CaP coatings on PE. The sample was scanned in a meandering way through the laser beam (FIG. 1). The setup had a beam size Sx×Sy (FIG. 1) between 280×280 μm² and 500×500 μm². The 20 ns laser pulses were fired at a rate of 50 Hz. The separation between the laser spots (dy) amounted to Sy/10. The horizontal distance between the tracks (dx in FIG. 2) was Sx/10, which resulted in 100 laser pulses on every irradiated part of the sample. The overlap in pulses ensured a homogeneous treatment across the material. Areas between 3×3 mm² and 10×10 mm² were irradiated. The energy per pulse was varied between 6 and 1000 mJ/cm² by changing the laser output or the focus. The irradiated samples were analyzed by XRD, RBS, and SEM.

### Temperature Calculation

The temperature in the coating due to a laser pulse was calculated by solving the heat equation:

$$\frac{δ T ( x , t )}{δ t} = κ \frac{{δ}^{2} T ( x , t )}{δ {x}^{2}}$$

In this equation, T is temperature, t is time, x is position, and κ is the thermal diffusivity, which is defined as

$$κ = \frac{k}{ρ C ρ} .$$

In this equation, k is the heat conductivity, Cp is the heat capacity, and p is the density. Numerical values for PE and HA are given in table 1. Because of unavailability of values for amorphous CaP, values for HA were used instead.

TABLE 1
Numerical values for parameters of HA and PE

| | HA | PE |
|---|---|---|
| k(W · m⁻¹ · K⁻¹⁾ | 1.26 | 0.44 |
| ρ(kg · m⁻³) | 3160 | 941-965 |
| Cp(J · kg⁻¹ · K⁻¹) | 772.9 | 2310 |
| k(m² · s⁻¹) | 5.2 · 10⁻⁷ | 2.0 · 10⁻⁷ |

The boundary conditions for the calculations were:

$$- k \frac{δ T}{δ x} ( 0 , t ) = \{ \begin{matrix}ɛ & t \leq {t}_{l} \\ 0 & t > {t}_{l}\end{matrix} \\ T ( ∞ , t ) = {T}_{0}$$

x=0 corresponds to the surface of the material, ε is the energy of the pulse per unit of time, T₀ is room temperature (298 K), and tₗ is the duration of the laser pulse (20 ns). The first condition imposes a heat flow through the surface when the laser is on, and no heat exchange when the laser is off. This assumption is allowed, as most of the laser light energy will be absorbed in the near-surface region, based on the optical transmission characteristics of the CaP coating that will be presented later (FIG. 3).

The second condition assumes room temperature deep inside the material. The initial condition was T (x, 0)=T₀. The heat equation was solved numerically by using Matlab (The MathWorks Inc., Natick Mass., USA). Because the difference in the κ's of HA and PE are not so large (table 1) and for computational simplicity, the substrate was assumed to be HA, instead of PE.

### Result

In FIG. 3, the optical transmission between 190 nm and 900 nm of uncoated and CaP coated sapphire is given. Because only one side is polished, the absolute transmission was only a few percent. Nevertheless, the CaP coated sapphire shows an optical absorption edge at ˜200 nm. In the figure, also the wavelengths of several excimer lasers are indicated. As a strong absorption of the laser light in the coating is required for laser crystallization, preferably the ArF (193 nm) or the F₂ laser (157 nm) may be suitable.

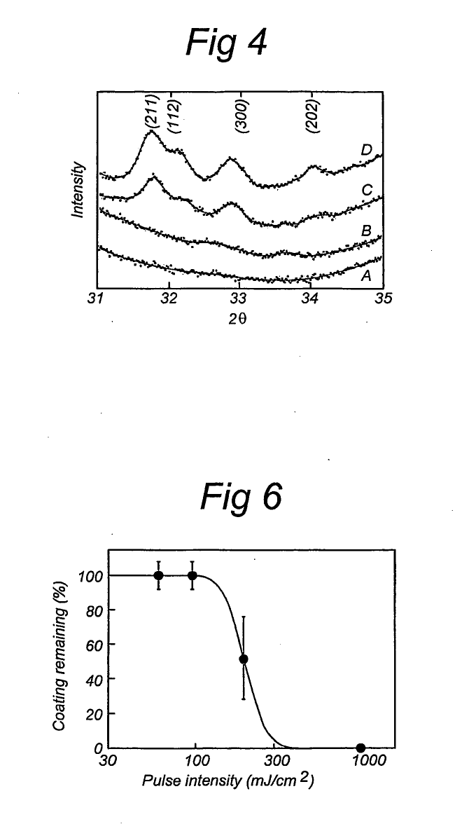

FIG. 4 shows the XRD spectra of the as-deposited CaP on PE (A) and fluorine laser treated CaP on PE (B, C, and D). The samples of spectra B, C, and D received laser pulses of 60, 96, and 200 mJ/cm², respectively. Spectra A and B do not show significant reflections, while spectra C and D show reflections that can be attributed to HA. The peaks in D are stronger than those in C.

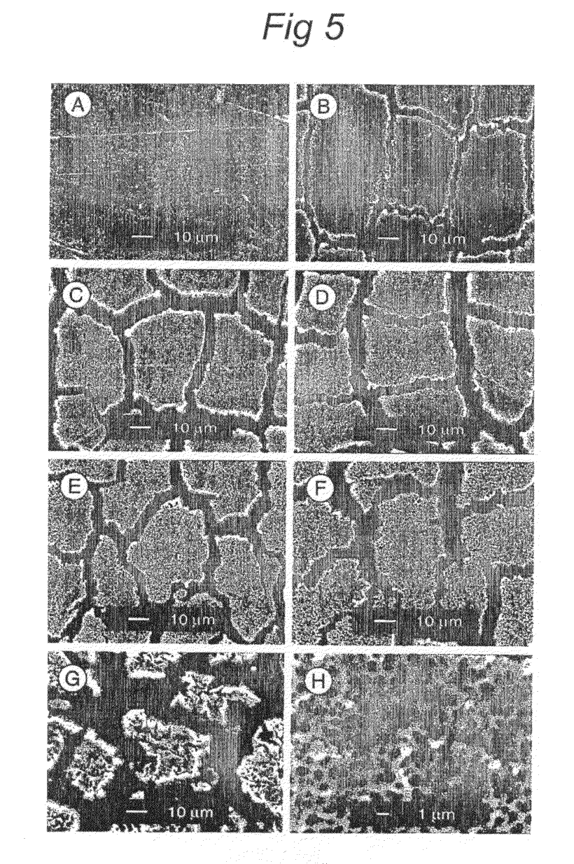

FIG. 5 shows SEM images of the F₂ laser treated CaP on PE. The energy per laser pulse for the images A-G was 0, 6, 10, 18, 49, 69, and 200 mJ/cm², respectively. Image H shows an enlargement of image F. With increasing energy per pulse, the cracks between the islands became larger. Besides, a porous structure appeared for the higherpulse energies (FIG. 5H). Further, with increasing dose, the shape of the islands became more irregular. Finally, it was noticed that for the highest doses, a height difference appeared, compared to the untreated material. By using a profilometer, the irradiated area was found to be up to several μm higher (data not shown).

FIG. 6 shows the remaining coating thickness of laser irradiated CaP ceramic, compared to the untreated material. Due to the laser treatment induced roughness (FIG. 5), it was difficult to determine the amount of coating that remained accurately. Nevertheless, a significant amount of the coating material was removed around a laser pulse intensity of 200 mJ/cm². For even higher pulse intensities (1000 mJ/cm²), all of the coating was removed, and the PE substrate obtained a light-brown color, indicating thermal degradation.

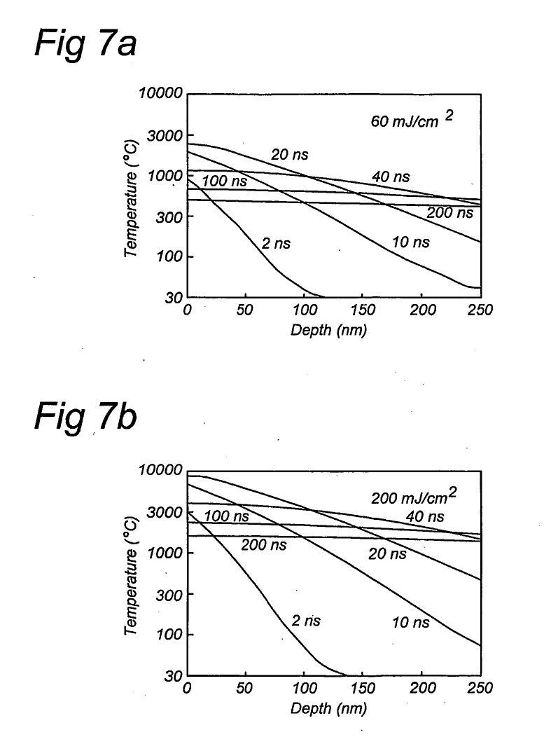

FIG. 7 shows the time-dependent temperature distributions in the CaP coating, for a laser pulse intensity of 60 mJ/cm² (A) and 200 mJ/cm² (B). The results are presented for 250 nm coating, the thickness that was used in the experiments. Until the time that the laser is switched off (20 ns), a strong increase in temperature is observed. Thereafter, the high temperatures diminish across the coating and into the interface.

### Discussion

The laser induced crystallization of CaP on PE was successful (FIG. 4). HA was observed on PE when a pulse power of at least 96 mJ/cm² was used. The higher laser pulse energy (200 mJ/cm²) gave a stronger crystallization The reason why a coating (partially) crystallizes, is the result of the temperature that the CaP coatings reached during irradiation, in combination with the time during which the coating was above the crystallization temperature (400-600° C., indicated in FIG. 7 by the dotted region). Probably, the time during which the CaP coating was at or above the crystallization temperature was too short to obtain crystallization in case of the 60 mJ/cm² pulses (FIG. 6A), while the temperature and duration were sufficient for crystallization at 96 and 200 mJ/cm². However, at 200 mJ/cm², part of the CaP coating was lost (FIG. 6). Probably, it evaporated from the coating, resulting from the extremely high temperatures that exist in the near-surface region during irradiation (FIG. 7B). Indeed, during laser irradiation at 200 mJ/cm², light emission above the substrate was observed, indicating the ejection of material. At a pulse intensity of 1000 mJ/cm², the light emission from evaporated material further increased, and almost no CaP coating was left on the substrate (FIG. 6).

SEM examination revealed that with increasing laser intensity, the cracks became broader, the islands obtained a more irregular shape, and porosity appeared in the material (FIG. 5). For the higher laser intensities the irradiated area raised several μm's compared to the unirradiated area. Probably, this is caused by a melting of the PE and the successive retraction into islands. Interestingly, uncoated PE that was irradiated with similar laser doses (not shown) did not show any cracks, porosity, nor a raised surface. This means that the CaP coating plays an active role in the formation of the morphology of the irradiated material. The mechanism behind the formation of this morphology is not clear though.

Because PE is a polymer with a fairly low melting temperature (141° C.), this work shows that a much broader range of polymers can be covered by a crystalline CaP coating, compared to a normal annealing treatment. For optimisation of the laser crystallization process a large number of parameters can be varied (pulse energy, pulse frequency, number of pulses, spot size). A great advantage of this technique is that it is in principle possible to crystallize large areas in a matter of seconds and a further advantage is the possibility for producing patterned crystalline regions.

### REFERENCES

1 L. L. Hench. Bioceramics. J. Am. Ceram. Soc., 81:1705-1728, 1998.
2 American Food and Drug Administration. Calcium phosphate (Ca-P) coating draft guidance for preparation of fda submissions for orthopedic and dental endosseous implants. Technical report, American Food and Drug Administration, 1997. Available on: www.fda.gov/cdrh/ode/33.html.
3 J. G. C. Wolke, K. de Groot, and J. A. Jansen. In vivo dissolution behavior of various rf magnetron sputtered ca-p coatings. J. Biomed. Mater. Res., 39:524-530, 1997.
4 J. G. C. Wolke, K. de Groot, and J. A. Jansen. Dissolution and adhesion behaviour of radio-frequency magnetron-sputtered Ca-P coatings. J. Mater. Sci., 33:3371-3376, 1998.
5 T. S. Chen and W. R. Lacefield. Crystallization of ion beam deposited calcium phosphate coatings. J. Mater. Res., 9:1284-1290, 1994.
6 K. van Dijk, H. G. Schaeken, J. G. C. Wolke, and J. A. Jansen. Influence of annealing temperature on rf magnetron sputtered calcium phosphate coatings. Biomaterials, 17:405-410, 1996.
7 M. Yoshinari, T. Hayakawa, J. G. C. Wolke, K. Nemoto, and J. A. Jansen. Influence of rapid heating with infrared radiation on RF magnetron-sputtered calcium phosphate coatings. J. Biomed. Mater. Res., 37:60-67, 1997.
8 J. Chen, J. G. C. Wolke, and K. de Groot. Microstructure and crystallinity in hydroxyapatite coatings. Biomaterials, 15:396-399, 1994.
9 K. A. Gross, V. Gross, and C. C. Berndt. Thermal analysis of amorphous phases in hydroxyapatite coatings. J. Am. Ceram. Soc., 81:106-112, 1998.
10 P. Layrolle, A. Ito, and T. Tateishi. Sol-gel synthesis of amorphous calcium phosphate and sintering into microporous hydroxyapatite bioceramics. J. Am. Ceram. Soc., 81:1421-1428, 1998.
11 C. F. Feng, K. A. Khor, S. W. K. Kweh, and P. Cheang. Thermally induced crystallization of amorphous calcium phosphate in plasma-spheroidised hydroxyapatite powders. Mater. Lett., 46:229-233, 2000.
12 C. M. Lopatin, V. B. Pizziconi, and T. L. Alford, Crystallization kinetics of sol-gel derived hydroxyapatite thin films. J. Mater. Sci.-Mater. Med., 12:767-773, 2001.
13 S. Raynaud, E. Champion, D. Bernache-Assollant, and P. Thomas. Calcium phosphate apatites with variable Ca/P atomic ratio I. synthesis, characterisation and thermal stability of powders. Biomaterials, 23:1065-1072, 2002.
14 S. Hontsu, M. Nakamori, H. Tabata, J. Ishii, and T. Kawai. Pulsed laser deposition of bioceramic hydroxyapatite thin films on polymer materials. Jpn. J. Appl. Phys. Part 2, 35:L1208-L1210, 1996.
15 S. Hontsu, M. Nakamori, N. Kato, H. Tabata, J. Ishii, T. Matsumoto, and T. Kawai. Formation of hydroxyapatite thin films on surface-modified polytetrafluoroethylene substrates. Jpn. J. Appl. Phys. Part 2, 37:L1169-L1171, 1998.
16 P. M. Smith, P. G. Carey, and T. W. Sigmon. Excimer laser crystalization and doping of silicon thin films on plastic substrates. Appl. Phys. Lett., 70:342-344, 1997.
17 L. Mariucci, A. Pecora, R. Carluccio, and G. Fortunato. Advances excimer laser crystallization techniques. Thin Solid Films, 383:39-44, 2001.
18 H. Hosono, M. Kurita, and H. Kawazoe. Excimer laser crystallization of amorphous indium-tin oxide thin films and application to fabrication of bragg gratings. Thin Solid Films, 351:137-140, 1999.
19 N. Asakuma, T. Fukui, M. Aizawa, M. Toki, H. Imai, and H. Hirashima. Ultravioletlaser-induced crystallization of sol-gel derived inorganic oxide films. J. Sol-Gel Sci. Technol., 19:333-336, 2000.
20 J. G. C. Wolke, J. M. A. de Blieck-Hogervorst, W. J. A. Dhert, C. P. A. T. Klein, and K. de Groot. Studies on the thermal spraying of apatite bioceramics. J. Therm. Spray Technol., 1:75-82, 1992.
21 M. Tanahasi T. Yao, T. Kokubo, M. Minoda, T. Miyamoto, T. Nakamura, and T. Yamamuro. Apatite coating on organic polymers by a a biomimetic process. J. Am. Ceram. Soc., 77:2805-2808, 1994.
22 P. Habibovic, F. Barrère, C. A. van Blitterswijk, K. de Groot, and P. Layrolle. Biomimetic hydroxyapatite coating on metal implants. J. Am. Ceram. Soc., 85:517-522,2002.
23 C. M. Cotell, D. B. Chrisey, K. S. Grabowski, J. A. Sprague, and C. R. Gossett. Pulsed laser deposition of hydroxylapatite thin films on Ti-6A1-4V. J. Appl. Biomater., 3:87-93, 1992.
24 R. K. Singh, F. Qian, V. Nagabushnam, R. Damodaran, and B. M. Moudgil. Excimer laser deposition of hydroxyapatite thin films. Biomaterials, 15:522-528, 1994.
25 X. Li, J. Weng, W. Tong, C. Zuo, X. Zhang, P. Wang, and Z. Liu. Characterization of hydroxyapatite film with mixed interface by Ar+ ion beam enhanced deposition. Biomaterials, 18: 1487-1493, 1997.
26 F. Z. Cui, Z. S. Luo, and Q. L. Feng. Highly adhesive hydroxyapatite coatings on titanium alloy formed by ion beam assisted deposition. J. Mater. Sci.-Mater. Med., 8:403-405, 1997.
27 K. van Dijk, J. Verhoeven, C. H. M. Marée, F. H. P. M. Habraken, and J. A. Jansen. Study of the influence of oxygen on the composition of thin films obtained by r.f. sputtering from a Ca5(PO4)3OH target. Thin Solid Films, 304:191-195, 1997.
28 K. van Dijk, H. G. Schaeken, C. H. M. Maŕee, J. Verhoeven, J. G. C. Wolke, F. H. P. M. Habraken, and J. A. Jansen. Influence of Ar pressure on r.f. magnetron-sputtered Ca5(PO4)3OH layers. Surf. Coat. Technol., 76-77:206-210, 1995.
29 K. van Dijk, H. G. Schaeken, J. G. C. Wolke, C. H. M. Marée, F. H. P. M. Habraken, J. Verhoeven, and J. A. Jansen. Influence of discharge power level on the properties of hydroxyapatite films deposited on Ti6A14V with RF magnetron sputtering. J. Biomed. Mater. Res., 29:269-276, 1995.
30 K. van Dijk, V. Gupta, A. K. Yu, and J. A. Jansen. Measurement and control of interface strength of RF magnetron-sputtered Ca-PO coatings on Ti-6A1-4V substrates using a laser spallation technique. J. Biomed. Mater. Res., 41:624-632, 1998.
31 Oxford Applied Research. Manual of the kaufmann ion source RF25. Crowley Mill, Witney, Oxon OX9 5TJ, United Kingdom.

1. Method for providing a polymeric implant object with a crystalline calcium phosphate (CaP) coating, said method comprising the step of irradiating a polymeric substrate having deposited thereon an amorphous CaP coating with laser light of <200 nm and 10-1000 mJ/cm².

2. Method according to claim 1 in which the irradiating with laser light <200 nm and 10-1000 mJ/cm² is carried out during deposition of a CaP coating onto a polymeric substrate.

3. Method according to claim 1 wherein the polymeric substrate comprises at least one selected from the group consisting of polyethylene (PE), poly(ethyleneterephthalate) (PET), polytetrafluoroethylene (PFTE), polystyrene (PS), poly-L-lactic acid (PLLA), polydimethylsiloxane (PDMS), polyimide (PI), polyglycolic acid (PGA), polypropylene fumarate (PPF) and polybutylterephthalate (PBT).

4. Method according to claim 1 wherein the CaP coating is deposited using any method suitable for depositing a CaP coating, said deposited CaP coating being amorphous.

5. Method according to claim 4, wherein the method suitable for depositing a CaP coating is selected from plasma spraying, biomimetic deposition, laser deposition, ion beam deposition and RF magnetron sputter deposition or combinations thereof, preferably RF magnetron sputter deposition.

6. Method according to claim 1 wherein the laser light is from a laser selected from the group consisting of F₂ and ArF.

7. Method according to claim 1, wherein the laser light has an energy of 10-500 mJ/cm².

8. Method according to claim 1 wherein the position of the laser relative to the object to be irradiated is controlled thereby creating a pattern of crystallisation on the irradiated object.

9. Polymeric implant object obtainable by the method according to claim 1.

10. Polymeric implant object according to claim 9, said object comprising a polymeric substrate having a crystalline CaP coating, said crystalline CaP coating having a thickness of at least 10 nm, but less than 1000 nm.

11. Polymeric implant object according to claim 9, wherein said implant is a fracture fixation plate, fixation screw, medullary nail, acetabular cup, or a guided tissue regeneration membrane.

12. Polymeric implant object according to claim 9, wherein said implant is of flexible polymeric material.

13. Method according to claim 2 wherein the polymeric substrate comprises at least one selected from the group consisting of polyethylene (PE), poly(ethyleneterephthalate) (PET), polytetrafluoroethylene (PFTE), polystyrene (PS), poly-L-lactic acid (PLLA), polydimethylsiloxane (PDMS), polyimide (PI), polyglycolic acid (PGA), polypropylene fumarate (PPF) and polybutylterephthalate (PBT).

14. Polymeric implant object according to claim 10, wherein said implant is a fracture fixation plate, fixation screw, medullary nail, acetabular cup, or a guided tissue regeneration membrane.

15. Polymeric implant object according to claim 10, wherein said implant is of flexible polymeric material.
